(19) United States
(12) Patent Application Publication
(10) Pub. No.: US 2026/0102157 A1
(43) Pub. Date: Apr. 16, 2026

(54) SOFT SUTURE CONSTRUCT WITH BIFURCATED SHEATH

(71) Applicant: Arthrex, Inc., Naples, FL (US)

(72) Inventors: Julia CUNY, Naples, FL (US); Trent BOYER, Bonita Springs, FL (US); Matthew JOHNSON, Bonita Springs, FL (US)

(21) Appl. No.: 19/354,559

(22) Filed: Oct. 9, 2025

Related U.S. Application Data
(60) Provisional application No. 63/706,478, filed on Oct. 11, 2024.

(51) Int. Cl. A61B 17/06

(52) U.S. Cl. CPC A61B 17/06166

(57) ABSTRACT

A suture construct may include a sheath, a repair strand, and an activation strand. The sheath may have a first distal end portion with a first opening, a second distal end portion with a second opening, and a tail portion. The repair strand may be in the sheath and have a length extending through the first distal end portion, wherein the repair strand is configured to engage a soft tissue. The activation strand may be in the sheath and extend through the first opening and the second opening to tether the first distal end portion and the second distal end portion. The activation strand may be configured to compress and anchor the sheath in a bone tunnel. The suture construct may be manufactured from a bifurcated structure and used in a method of repairing soft tissue.

### PRIORITY

This application claims the benefit of priority to U.S. Provisional Application No. 63/706,478, filed October 11, 2024, the entire disclosure of which is incorporated herein.

### TECHNICAL FIELD

This disclosure relates generally to a soft suture construct, and more specifically, directed to a suture construct having a bifurcated sheath.

### BACKGROUND

When soft tissue, such as a ligament or a tendon, tears or becomes detached from a bone, surgery is usually required to reattach or reconstruct the tissue. Techniques and devices have been developed generally involve securing an anchor in a hole provided in the bone tissue and tying the soft tissue with suture to an anchor. The bone anchor can be a hard anchor or a soft anchor.

### SUMMARY

The present inventors recognize that hard suture anchors often require substantial bone removal for securing the anchor due to the bulky nature of the hard suture anchors. Hard anchors also typically require direct access and rotating the bone anchor to be secured in place with a threaded engagement, which can be difficult to achieve in minimally invasive procedures. Furthermore, soft anchors can involve complex braiding patterns that can be difficult to manufacture.

Thus, a first aspect of the present disclosure is directed to a suture construct including: a sheath having a first distal end portion with a first opening, a second distal end portion with a second opening, and a tail portion; a repair strand in the sheath and having a length extending through the first distal end portion, wherein the repair strand is configured to engage a soft tissue; and an activation strand in the sheath and extending through the first opening and the second opening to tether the first distal end portion and the second distal end portion, wherein the activation strand is configured to compress and anchor the sheath in a bone tunnel.

In some embodiments, the suture construct may include one or more of the following features. The length of the repair strand extends through the first opening. The activation strand is a closed loop. The activation strand extends proximally from the first distal end portion and the second distal end portion, through the tail portion, and through a third opening in the tail portion. The sheath is formed of a plurality of woven, knitted, or braided threads, and the threads of the tail portion bifurcate to form the first distal end portion and the second distal end portion. A first length of the tail portion has a first thread count, the first distal end portion and/or the second distal end portion have a second thread count, and the first thread count is greater than the second thread count. A second length of the tail portion has a third thread count, and the first thread count is greater than the third thread count. The repair strand has a second length extending through the second distal end portion. The repair strand is received in the tail portion and bifurcates to form the length and the second length. The repair strand is fixed to a portion of the sheath. A shuttle is associated with the repair strand. The shuttle is spliced through a portion of the length of the repair strand. The portion of the length of the repair strand is disposed entirely within the sheath. The shuttle has a fixed loop at a distal end.

A second aspect of the present disclosure is directed to a method of forming a suture construct from a structure braided with a pattern having a non-bifurcated segment and a bifurcated segment, the method comprising: trimming a first length of the bifurcated segment to form a first distal end portion having a first opening; trimming a second length of the bifurcated segment to form a second distal end portion having a second opening; inserting a repair strand into the sheath by extending a length of the repair strand through the first distal end portion; and inserting an activation strand into the sheath by extending the activation strand through the first opening and the second opening to tether the first distal end portion and the second distal end portion.

In some embodiments, the method may include one or more of the following features. The length of the repair strand extends through the first opening. The pattern has a second bifurcated segment, and the method further include: trimming a first length of the second bifurcated segment and a second length of the second bifurcated segment; and splicing the second length of the second bifurcated segment into the first length of the second bifurcated segment. The method may include trimming the first length of the second bifurcated segment or the second length of the second bifurcated segment after the splicing. The method may include inserting a second length of the repair strand into the sheath by extending the second length of the repair strand through the second distal end portion. The method may include splicing a shuttle through a portion of the length of the repair strand, wherein the portion of the repair strand is disposed entirely within the sheath when the repair strand is in the sheath.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates an embodiment of a suture construct according to the present disclosure.

FIG. 2 illustrates a cross-sectional view of the suture construct of FIG. 1.

FIGS. 3A-C illustrate an exemplary method of tissue repair using the suture construct of FIGS. 1 and 2 according to the present disclosure.

FIG. 4A-C illustrate an exemplary method of making the suture construct of FIGS. 1 and 2 according to the present disclosure.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

The present disclosure relates to a suture construct, methods of using the suture construct for repairing tissue, and methods of making the suture construct. The suture construct of the present disclosure may be an all-suture implant. The suture construct may be designed to facilitate anchoring and securing a soft tissue to bone, for example through a portal in a minimally invasive procedure. The suture construct may be double-loaded allowing for two repair suture ends in a single anchor pass. In some embodiments, the suture construct may facilitate inline repairs in a knotless technique with one or more pre-loaded shuttles. The all-suture design of the suture construct may allow minimal bone removal with a smaller drill pin. The design of the suture construct of the present disclosure may further allow for a simplified method of repairing tissue as compared to conventional repair techniques. For example, a simplified inserter may be used to the suture construct for being pulled instead of pushed into place. In an exemplary application, the suture construct may be used to repair a meniscal root repair by anchoring the same in a transtibial tunnel. The suture construct may eliminate the need for posterior knee portals and may allow for direct visualization of the repair site. Furthermore, the suture construct of the present disclosure may be easily manufactured with a bifurcated pattern.

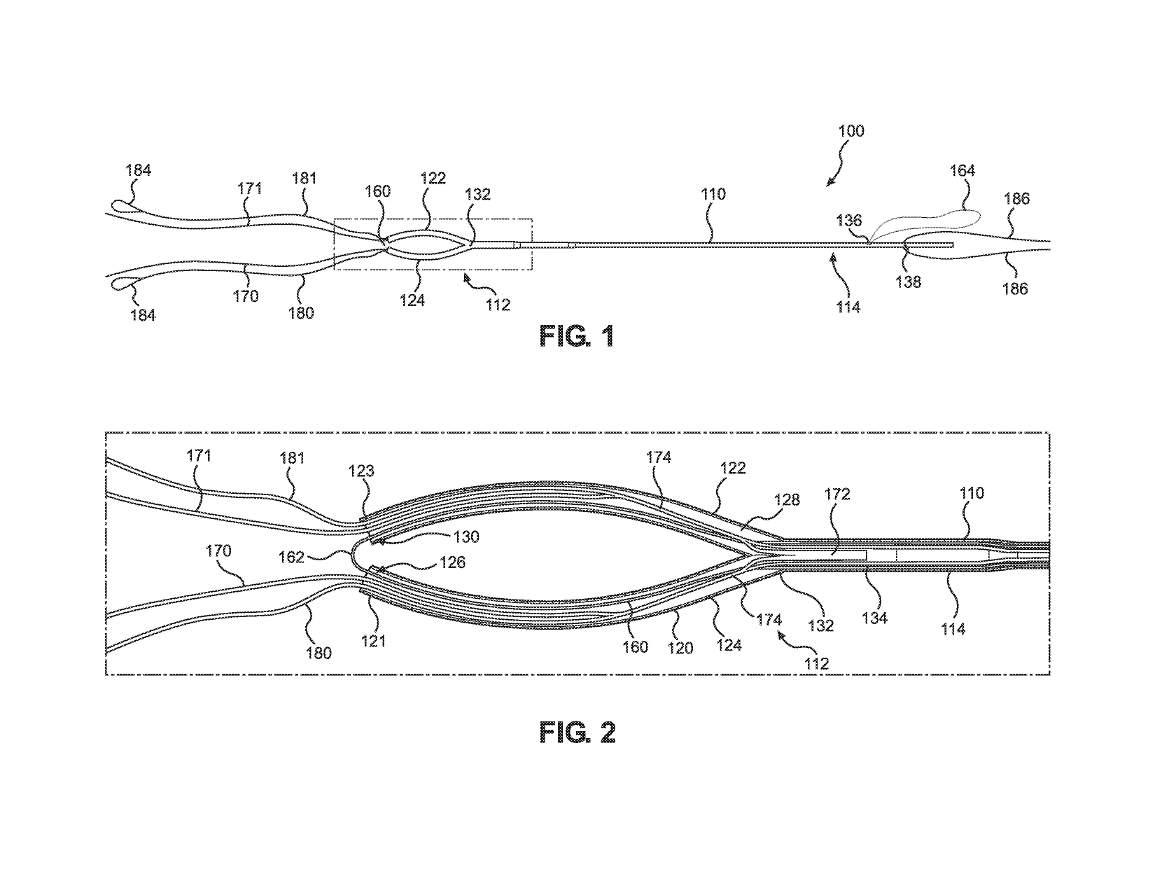

FIGS. 1 and 2 illustrate a suture construct 100. The suture construct 100 may include a sheath 110, an activation strand 160, at least one repair strand 170, 171, and at least one shuttle 180, 181. The sheath 110 may have at least one lumen that receives the activation strand 160, the at least one repair strand 170, 171, and the at least one shuttle 180, 181. The activation strand 160 may be pulled to compress the sheath 110 to anchor the suture construct 100 in a bone tunnel. The at least one repair strand 170, 171 may be configured to engage a soft tissue to secure the soft tissue to the bone. In some embodiments, the one or more shuttles 180, 181 may be provided to thread the at least one repair strand 170, 171 to be spliced into itself and provide a knotless mechanism to secure the soft tissue. In some embodiments, the one or more shuttles 180, 181 may be omitted.

The sheath 110 may have a head portion 112 at a distal end and a tail portion 114 that extends from the head portion 112 to a proximal end. The head portion 112 may include a first end portion 120 and a second end portion 122 distal of a bifurcation 132 of the tail portion 114. The first end portion 120 and the second end portion 122 may be spaced apart or disconnected distal of the bifurcation 132 of the tail portion 114. For example, a first terminal end 121 of the first end portion 120 may be spaced apart or disconnected from a second terminal end 123 of the second end portion 122. The first end portion 120 may have a first lumen 124 with a first opening 126, and the second end portion 122 may have a second lumen 128 with a second opening 130. The first opening 126 may be through the terminal end 121 end of the first end portion 120 or may be spaced from the first terminal end 121 (e.g., through a transverse wall portion). Similarly, the second opening 130 may be through the second terminal end 123 of the second end portion 122 or may be spaced from the second terminal end 123 (e.g., through a transverse wall portion). Further discussion of exemplary features of the suture construct 100 and the openings 126, 130 is provided in U.S. Pat. 9,622,738, the entire disclosure of which is expressly incorporated herein by reference. The first lumen 124 and the second lumen 128 may be in communication with a tail lumen 134 through the tail portion 114. The tail lumen 134 may extend proximally from the bifurcation 132 to one or more proximal openings 136, 138 in the tail portion 114.

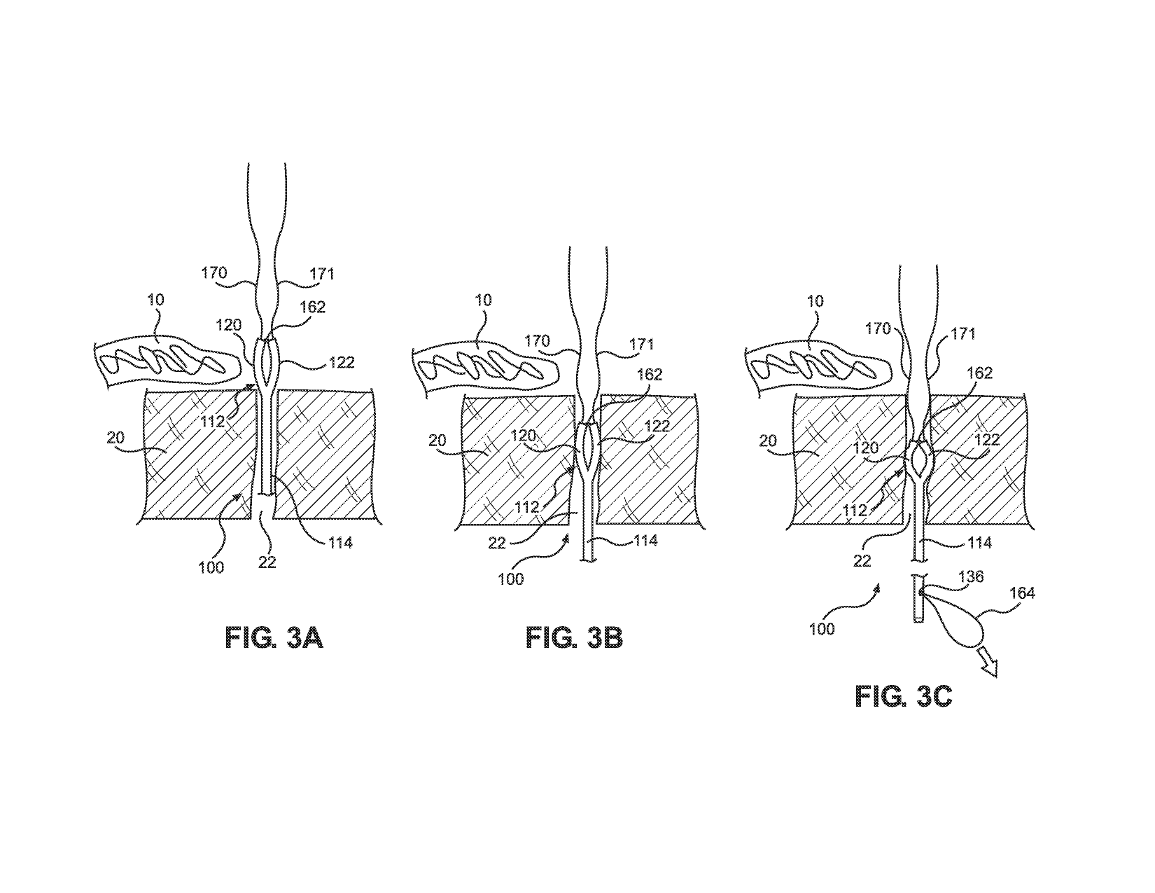

The sheath 110 may be formed of a flexible material such that the head portion 112 may be longitudinally compressed or bunched. The head portion 112 may be compressed or bunched between an uncompressed configuration (e.g., FIG. 3A, B) and a longitudinally compressed configuration (e.g., FIG. 3C) in order to set or anchor the sheath 110 inside of the bone tunnel. In some embodiments, the sheath 110 may be elastic. The sheath 110 may be formed of a plurality of strands, such as yarns, fibers, and/or filaments that are woven, braided, and/or knitted into the head portion 112 and the tail portion 114. The sheath 110 may be made of at least one ultrahigh molecular weight polyethylene (UHMWPE) strand, at least one polyester strand, and/or at least one elastic strand. For example, in some embodiments, the sheath 110 may be constructed of a plurality of UHMWPE strands, for example, entirely of UHMWPE strands. In some embodiments, the sheath 110 may be constructed of a plurality of polyester strands, for example, entirely of polyester strands. In some embodiments, the sheath 110 may be constructed of at least one UHMWPE strand and at least one polyester strand. In some embodiments, the sheath 110 may be constructed of at least one UHMWPE strand and at least one elastic strand. In some embodiments, the sheath 110 may be constructed of at least one polyester and at least one elastic strand. In some embodiments, the sheath 110 may be constructed of at least one UHMWPE strand, at least one polyester strand, and at least one elastic strand. The elastic strand may be composed of elastane. The at least elastic strand may provide elasticity, while the at least one UHMWPE strand and/or the at least one polyester stand may add strength and limit the elongation of the sheath 110. The sheath 110 may be loosely braided to facilitate splicing of the activation strand 160, the at least one repair strand 170, 171, and/or the at least one shuttle 180, 181. The loose braiding of the sheath 110 may, additionally or alternatively, facilitate longitudinal compression of the end portions 120, 122 of the head portion 112.

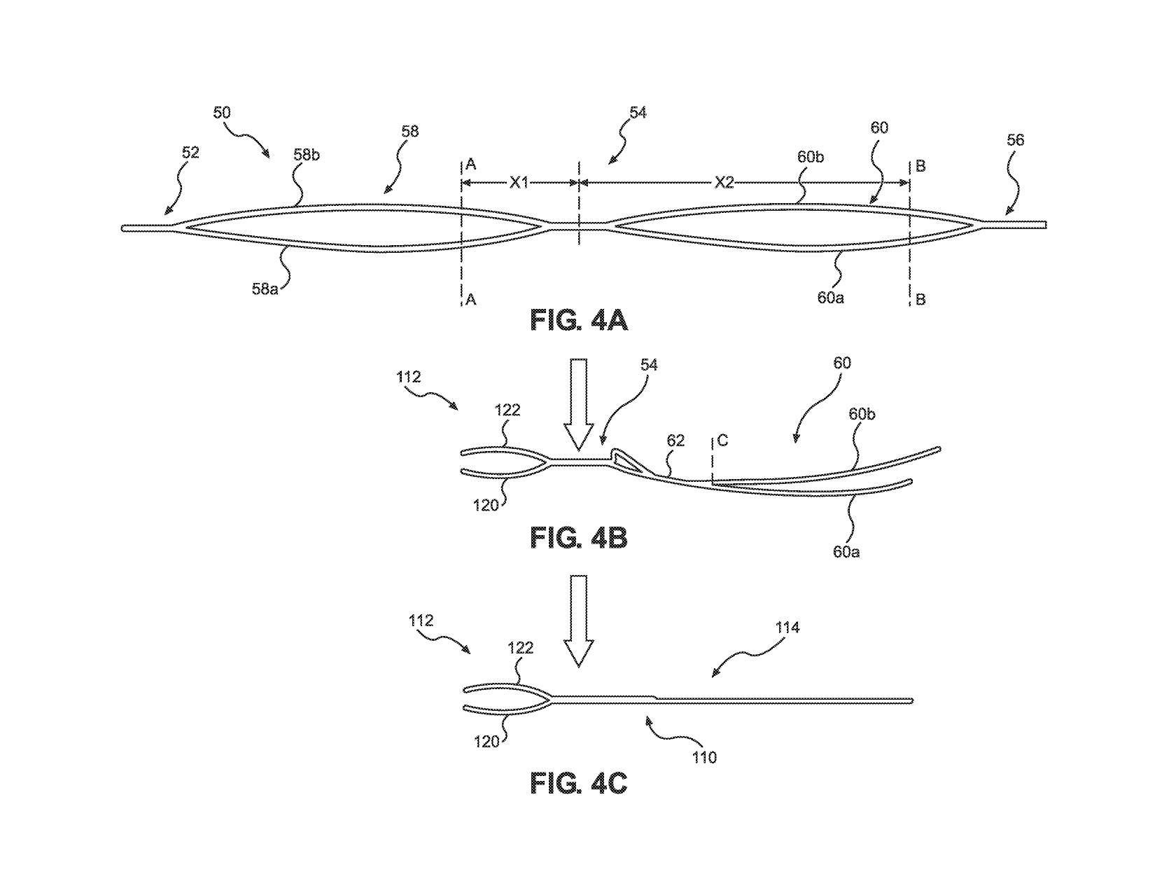

The sheath 110 may be a single unitary structure such that the head portion 112 and the tail portion 114 are formed continuously. As further discussed with reference to FIGS. 4A-C, the strands may be braided into a pattern having at least one non-bifurcated segment and at least one bifurcated segment. The first end portion 120 may be formed by trimming a first length of a bifurcated segment and the second end portion 122 may be formed by trimming a second length of the same bifurcated segment. At least a first length of the tail portion 114 may be formed from a non-bifurcated segment. Thus, the first length of the tail portion 114 may have a first thread count, and the first end portion 120 and/or the second end portion 122 may have a second thread count. The first thread count may be greater than the second thread count. The first end portion 120 and/or the second end portion 122 may have the same or different thread count. For example, the first thread count of the tail portion 114 may be 32 strands, and the second thread count of the first end portion 120 and the second end portion 122 may be 16 strands. The reduced number of threads in the end portions 120, 122 may allow the activation strand 160 to compress or bunch the end portions 120, 122 without substantial compression of the first length of the tail portion 114. The tail portion 114 may have a second length composed of a second bifurcated segment, as further discussed with reference to FIGS. 4A-C. The second length of the tail portion 114 may be formed of a single length of the second bifurcated segment, such that the second length of the tail portion 114 may have a third thread count less than the first thread count of the first length of the tail portion 114. For example, the third thread count may be the same as the second thread count of each of the end portions 120, 122.

The activation strand 160 may be a flexible strand that extends through the sheath 110. Returning to FIGS. 1 and 2, the activation strand 160 may have a distal portion 162 looped through the head portion 112 and a proximal portion 164 that may be manipulated by the user to compress the head portion 112. A first length of the distal portion 162 may extend through the first lumen 124 of the first end portion 120. A second length of the distal portion 162 may extend through the second lumen 128 of the second end portion 122. The distal portion 162 may extend through the first opening 126 of the first end portion 120 and the second opening 130 of the second end portion 122. The distal portion 162 may extend across a space between the first terminal end 121 and the second terminal end 123 to form a loop that physically connects or tethers the first end portion 120 and the second end portion 122. FIGS. 1 and 2 illustrate the activation strand 160 extending through the same openings 126, 130 as the at least one strand 170, 171 and the shuttles 180, 181. This configuration may simplify loading of the sheath 110. However, it is also contemplated that the activation strand 160 may extend through different openings than the at least one of the at least one repair strand 170, 171 and/or the shuttles 180, 181. In some embodiments (not shown), the activation strand 160 may enter and exit one or more times along the length of at least one of the end portions 120, 122, at the same or different locations. For example, the activation strand 160 may be spliced into and out of openings at different locations along the length of the wall of each of the end portions 120, 122 to more evenly distribute the bunching along its length.

The first length and the second length of the activation strand 160 may extend proximally through the head portion 112, past the bifurcation 132, and into the tail lumen 134. As illustrated in FIG. 1, the activation strand 160 may have a proximal portion 164 that extends proximally through the tail lumen 134 and out of the one or more proximal openings 136 to be manipulated or pulled by the user to compress the head portion 112. In some embodiments, the activation strand 160 may be a closed loop forming a closed proximal end of the proximal portion 164. In some embodiments, a first length of the proximal portion 164 may extend or be spliced out of a first proximal opening 136 and a second length of the proximal portion 164 may extend or be spliced out of a second proximal opening 136. Extending the first and second lengths of the proximal portion 164 out of different proximal openings 136 may help prevent the proximal portion 164 from being pulled into the tail lumen 134. In some embodiments, the first length and the second length of the proximal portion 164 may extend or be spliced out of the same proximal opening 136. In some embodiments, the activation strand 160 may not be a closed loop and define two free ends at the proximal portion 164.

The at least one repair strand 170, 171 may be received in the sheath 110, extend out of the head portion 112, and be configured to engage the soft tissue. The at least one repair strand 170, 171 may have free ends extending from at least one of the openings 126, 130 for use in the tissue repair procedure. As illustrated in FIGS. 1 and 2, the at least one repair strand may have a first length 170 received in the first lumen 124 of the first end portion 120 and extend out of the first opening 126 and a second length 171 received in the second lumen 128 of the second end portion 122 and extend out of the second opening 130. The at least one repair strand 170, 171 may be configured to be passed through and/or around the soft tissue to secure the soft tissue to the bone through knotted or knotless fixtures. The at least one repair strand 170, 171 may be pre-loaded in the sheath 110 during manufacturing, prior to insertion into the body.

In some embodiments, the first length 170 and the second length 171 may be formed from the same repair strand. As further illustrated in FIG. 2, the repair strand 170, 171 may have a proximal portion 172 fixed to the sheath 110 in the tail lumen 134. The first length 170 and the second length 171 may be formed from bifurcated extensions of the proximal portion 172. Fixing the at least one repair strand 170, 171 to the sheath 110 may improve the ability to tension the at least one flexible repair strand 170, 171, such as while performing a bridging technique. In some embodiments, the at least one repair strand 170, 171 may be fixed to the sheath 110 by being spliced or looped through a wall of the sheath 110, such as at the proximal portion 172. In some embodiments, the at least one repair strand 170, 171 may be fixed to the sheath 110 by one or more stitches extending through the proximal portion 172 and the sheath 110, such as at the proximal portion 172. In some embodiments, the at least one repair strand 170, 171 may be fixed to the sheath 110 with a glue or an adhesive, such as at the proximal portion 172. Further discussion of exemplary features of fixing the at least one repair strand 170, 171 to the sheath 110 are provided in U.S. Patent No. 9,622,738, the entire disclosure of which is expressly incorporated herein by reference. In some embodiments, the first length 170 and the second length 171 may be formed of separate repair strands. Additionally or alternatively, the at least one repair strand 170, 171 may not be fixed to the sheath 110 and slide within the lumen of the sheath 110.

In some embodiments, as further illustrated in FIGS. 1 and 2, the at least one shuttle 180, 181 may be provided and associated with the at least one repair strand 170, 171. The at least one shuttle 180, 181 may be spliced through a portion 174 of the at least one repair strand 170, 171. The spliced configuration of the shuttle 180, 181 may allow the soft tissue to be fixed with a knotless self-locking mechanism and allow the user to control the tension of the repair strand 170, 171 by actuating a proximal portion 186 of the at least one shuttle 180, 181. The at least one shuttle 180, 181 may be preloaded into the sheath 110 by being spliced into the at least one repair strand 170, 171 prior to inserting into the at least one repair strand 170, 171 into the sheath 110. As illustrated, each shuttle 180, 181 may have a loop 184 (e.g., at a distal end) configured to receive a free end of one of the repair strands 170, 171.

A first shuttle 180 may extend proximally from its distal end into the first opening 126 and into the first lumen 124 of the first end portion 120. The first shuttle 180 may extend or be spliced into and out of a portion 174 of the first length of repair strand 170, such that the entire spliced portion 174 is inside of the sheath 110 (e.g., inside of the first lumen 124 and/or the tail lumen 134). The first shuttle 180 may extend proximally through the tail lumen 134 with the proximal portion 186 extending through the proximal opening 138 and out of the sheath 110 to be manipulated by the user. Thus, after passing a free end of the first length of repair strand 170 through the loop 184, the user may pull the proximal portion 186 of the first shuttle 180 to pull the free length of repair strand 170 into the first opening 126 and into the spliced portion 174 of the first repair strand 170 such that it doubles on itself inside of the sheath 110. The spliced portion 174 may frictionally engage the free of the first length of repair strand 170 inside of the spliced portion 174 of the first length of repair strand 170 to form a cinched loop knotless construct. Continued pulling of the first shuttle 180 may pull the first shuttle 180 out of the spliced portion 174 of the first length of repair strand 170 and disengage the free end of the first repair strand 170 from the loop 184. The first shuttle 180 may then be pulled out of the proximal opening 138 of the sheath 110 for removal from the surgical site.

Similarly, a second shuttle 181 may extend proximally from its distal end into the second opening 130 and into the second lumen 128 of the second end portion 122. The second shuttle 181 may extend or be spliced into and out of a portion 174 of the second length of repair strand 171, such that the entire spliced portion 174 is inside of the sheath 110 (e.g., inside of the second lumen 128 and/or the tail lumen 134). The second shuttle 181 may extend proximally through the tail lumen 134 with the proximal portion 186 extending out of the sheath 110 through the same or different proximal opening 138 as the first shuttle 180 to be manipulated by the user. Thus, after passing a free end of the second length of repair strand 171 through the loop 184, the user may pull the proximal portion 186 of the second shuttle 181 to pull the second length of repair strand 171 into the second opening 130 and into the spliced portion 174 of the second length of repair strand 171 such that it doubles on itself inside the sheath 110. The spliced portion 174 may frictionally engage the free of the second length of repair strand 171 inside of the spliced portion 174 of the second length of repair strand 171 to form a cinched loop knotless construct. Continued pulling of the second shuttle 181 may pull the second shuttle 181 out of the spliced portion 174 of the second length of repair strand 171 and disengage the free end of the second length of repair strand 171 from the loop 184. The second shuttle 181 may then be pulled out of the proximal opening 138 of the sheath 110 for removal from the surgical site.

The at least one repair strand 170, 171 may be a high-strength suture, formed from at least one strand of UHMWPE that can easily be spliced. In some embodiments, the at least one repair strands 170, 171 may be a FiberWire® suture, produced by Arthrex and disclosed in U.S. Pat. No. 6,716,234, the entire disclosure of which is incorporated herein by reference. FiberWire® suture may be formed of UHMWPE braided with at least one other fiber to form lengths of suture material. The FiberWire® suture may include a core within a hollow braided construct, the core being a twisted yarn of UHMWPE. Additional non-limiting alternatives of the at least one repair strands 170, 171 include TigerWire® suture, FiberChain® suture, or TightRope® suture (all produced by Arthrex), although other cored or coreless sutures may be utilized. Additionally or alternatively, the at least one repair strands 170, 171 may be a flat suture tape, such as FiberTape® produced by Arthrex. In some embodiments, the suture construct 100 may include one or more secondary fixation devices, such as a button attached to the at least one repair strands 170, 171. The at least one shuttle 180, 181 may be a FiberLink™ suture, a TigerLink™ suture (both produced by Arthrex), or a Nitinol loop.

FIGS. 3A-3C illustrate an exemplary method of securing a soft tissue 10 to a bone tissue 20 by anchoring the suture construct 100 in a tunnel 22 through the bone tissue 20. The suture construct 100 may be used in a variety of techniques to attach the soft tissue 10 to the bone tissue 20. In one example, the soft tissue 10 may be a meniscal tissue and the bone tissue may be a tibia, and the method may be implemented in a transtibial meniscal root repair. The bone tunnel 22 may be formed or drilled transversely through the bone tissue 20 by a drill pin of reduced size. The user may initially pass a suture threader (e.g., a nitinol wire, not shown) through a first access opening of the body (not shown), through the bone tunnel 22, and then through a second access opening of the body (not shown). The first and second access openings may be anterior, such that no posterior access openings would be required in the procedure. The first access opening may be defined by a pin drilled through a transtibial tunnel, where the pin may be cannulated to receive the suture threader. The second access opening may be defined by a button cannula at an exit of the bone tunnel 22.

The user may engage the suture construct 100 at the tail portion 114 with the suture threader outside of the second access opening. The user may pull the suture threader from the first access opening. The user may thus pass the suture construct 100 through the second access opening in a retrograde fashion and into the bone tunnel 22. Thus, as illustrated in FIGS. 3A-B, the suture construct 100 may be passed into the bone tunnel 22 such that the tail portion 114 is passed into the bone tunnel 22 before the head portion 112. As illustrated in FIG. 3B, the suture construct 100 may be placed such that the head portion 112 is just below a surface of the bone tissue 20 (e.g., a tibial plateau) and the tail portion 114 of the sheath 110 extends through the bone tunnel 22. The bone tunnel 22 may slightly compress the end portions 120, 122 laterally. The at least one repair strands 170, 171 and the at least one shuttle 180, 181 may extend from the bone tunnel 22 to be available for use in securing the soft tissue 10. The suture construct 100 may have a length such that the proximal end of the tail portion 114 may extend from the bone tunnel 22 through the first access opening to be accessed by the user. Similarly, the distal ends of the at least one repair strands 170, 171 and the at least one shuttle 180, 181 may extend from the body through the second access opening to be accessed by the user. The at least one shuttle 180, 181 are not shown in FIGS. 3A-C for clarity purposes.

As illustrated in FIG. 3C, once the suture construct 100 is pulled into the body and positioned in the bone tunnel 22, the proximal portion 164 of the activation strand 160 may be pulled or tensioned to bunch or compress the head portion 112 of the sheath 110. The activation strand 160 may be pulled in the retrograde direction to bunch the head portion 112 and anchor the fixation device within the bone tunnel 22. Pulling the proximal portion 164 may pull the distal portion 162 of the activation strand 160 to shorten and laterally expand the end portions 120, 122. In the bunched configuration, the head portion 112 may frictionally engage the inner surface of the bone tunnel 22 and/or create an interference fit to anchor the sheath 110 in the bone tunnel 22. A clamp may be temporarily placed on the tail portion 114 to ensure that the suture construct 100 is anchored.

With the suture construct 100 anchored in the bone tunnel 22, the soft tissue 10 may be repaired and secured to the bone tissue 20 with direct visualization. The repair lengths 170, 171 may remain outside of the bone tunnel 22 and available for use in securing the soft tissue 10. The first repair length 170 may be loaded onto a suture passer (e.g., a Scorpion^{TM} as produced by Arthrex, not shown) to be passed through a first portion of the soft tissue 10. The first repair length 170 may then be threaded through the loop 184 of the first shuttle 180. The second repair length 171 may be loaded onto the suture passer to be passed through a second portion of the soft tissue 10. The second repair length 171 may then be threaded through the loop 184 of the second shuttle 181. The clamp may be released from the tail portion 114, and each of the proximal portions 186 of the shuttles 180, 181 may be pulled to retract each of the repair length 170, 171 into the respective spliced portions 174 to form the knotless anchor. Each of the activation strand 160 and the shuttles 180, 181 may be independently tensionable to ensure that the soft tissue 10 is securely tensioned. Once the soft tissue 10 is secured to the bone tissue 20, the tail portion 114 may be trimmed (e.g., at the tibial cortex) to complete the soft tissue repair.

FIGS. 4A-C illustrate an exemplary process of making the suture construct 100 of FIGS. 1 and 2 according to the present disclosure. As illustrated, the suture construct 100 may be formed by cutting a length of a long fibrous structure 50. The structure 50 may be formed of a plurality of strands, such as yarns, fibers, and/or filaments that are woven, braided, and/or knitted, as discussed with reference to the suture construct 100. As further illustrated, the structure 50 may be formed of a pattern including repeating non-bifurcated segments 52-56 and bifurcated segments 58, 60. Each of the bifurcated and non-bifurcated segments 52-60 may be formed with a lumen as discussed herein. The structure 50 may have any number of bifurcated and non-bifurcated segments 52-60 to form a length that may be trimmed to any number of suture constructs 100. Each of the bifurcated segments 58, 60 may be formed of a first length 58a, 60a and a second length 58b, 60b.

The structure 50 may be formed with a braiding machine having carriers configured to pass the strands through one or more tracks around one or more axes (not shown). The braiding machine may have a first mode to form the non-bifurcated segments and a second mode to form the bifurcated segments 58, 56. In the first mode, the braiding machine may pass all of the strands in a single path of travel around a common axis to form the non-bifurcated segments 52-56. In the second mode, the braiding machine may pass a first subset strands in a first path of travel around a first axis to form the first length 58a, 60a of the bifurcated segments 58, 56 and a second subset of the strands around a second axis in a second path of travel to form the second length 58b, 60b of the bifurcated segments 58, 56. For example, the braiding machine may have a bar that passes between two different sets of carriers to transition the braiding machine from the first mode to the second mode. The bar may have channels that when present divides the single path of travel of the first mode into the two separate paths of the second mode to form the bifurcated segments 58, 56.

Thus, the non-bifurcated segments 52-56 may be formed of a greater number of strands than each of the first length 58a, 60a and the second length 58b, 60b of the bifurcated segments 58, 60. In some embodiments, the non-bifurcated segments 52-56 may be formed of twice as many strands as each of the first length 58a, 60a and the second length 58b, 60b. For example, the non-bifurcated segments 52-56 may be formed of 32 strands, and each of the first length 58a, 60a and the second length 58b, 60b may be formed of 16 strands.

Thus, as further illustrated in FIGS. 4A-C, the structure 50 may have a first bifurcated segment 58 and a second bifurcated segment 60. The first bifurcated segment 58 may extend between and connect a first non-bifurcated segment 52 and a non-bifurcated segment 54, and the second bifurcated segment 60 may extend between and connect the non-bifurcated segment 54 and a third non-bifurcated segment 56.

The structure 50 may be cut at one or more locations to form the sheath 110. A first cut of the first length 58a and the second length 58b of the first bifurcated segment 58 may be along a first axis A to form the end portions 120, 122. The first cut may separate the end portions 120, 122 from the remaining length of the first bifurcated segment 58 and the first non-bifurcated segment 52. The first cut may form the openings 126, 130 at the terminal ends 121, 123 of the end portions 120, 122.

A second cut of the second length 60a and the second length 60b of the second bifurcated segment 60 may be along a second axis B. The second cut may separate a portion of the second lengths 60a, 60b from the remaining length of the second bifurcated segment 60 and the third non-bifurcated segment 56. The first axis A may be at a first distance X1 from a center of the non-bifurcated segment 54, and the second axis B may be at a second distance X2 from the center of the non-bifurcated segment 54, where the second distance X2 may be greater than the first distance X1.

As illustrated in FIG. 4B, the second length 60b of the second bifurcated segment 60 may be spliced into and out of the first length 60a at a distance from the non-bifurcated segment 54 (less than the second distance X2) to form a buried tail 62. The second length 60b may then be tensioned to pull the second length 60b along the first length 60a. The first length 60a or the second length 60b may be cut along a third axis C at or near the location that the second length 60b exits the spliced region of the first length 60a, while the remaining first length 60a or second length 60b may form a length of the tail portion 114. The buried tail 62 may be fixed inside of the first length 60a, for example through welding and/or an adhesive. As illustrated in FIG. 4C, the resultant structure formed by the non-bifurcated segment 54 and the buried tail 62 may be a non-bifurcated length of the tail portion 114 of the sheath 110.

After the sheath 110 is formed, the activation strand 160 may be spliced into the sheath 110. The first shuttle 180 may be spliced into the first length 170 of the at least one repair strand, and the second shuttle 181 may be spliced into the second length 171 of the at least one repair strand. The at least one repair strand 170, 171, the first shuttle 180, and the second shuttle 181 may be spliced into the sheath 110 and fixed to the sheath 110, as discussed herein.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A suture construct comprising:
a sheath having a first distal end portion with a first opening, a second distal end portion with a second opening, and a tail portion;
a repair strand in the sheath and having a length extending through the first distal end portion, wherein the repair strand is configured to engage a soft tissue; and
an activation strand in the sheath and extending through the first opening and the second opening to tether the first distal end portion and the second distal end portion, wherein the activation strand is configured to compress and anchor the sheath in a bone tunnel.

2. The suture construct of claim 1, wherein the length of the repair strand extends through the first opening.

3. The suture construct of claim 1, wherein the activation strand is a closed loop.

4. The suture construct of claim 1, wherein the activation strand extends proximally from the first distal end portion and the second distal end portion, through the tail portion, and through a third opening in the tail portion.

5. The suture construct of claim 1, wherein the sheath is formed of a plurality of woven, knitted, or braided threads, and the threads of the tail portion bifurcate to form the first distal end portion and the second distal end portion.

6. The suture construct of claim 5, wherein a first length of the tail portion has a first thread count, the first distal end portion and/or the second distal end portion have a second thread count, and the first thread count is greater than the second thread count.

7. The suture construct of claim 6, wherein a second length of the tail portion has a third thread count, and the first thread count is greater than the third thread count.

8. The suture construct of claim 1, wherein the repair strand has a second length extending through the second distal end portion.

9. The suture construct of claim 8, wherein the repair strand is received in the tail portion and bifurcates to form the length and the second length.

10. The suture construct of claim 1, wherein the repair strand is fixed to a portion of the sheath.

11. The suture construct of claim 1, further comprising a shuttle associated with the repair strand.

12. The suture construct of claim 11, wherein the shuttle is spliced through a portion of the length of the repair strand.

13. The suture construct of claim 12, wherein the portion of the length of the repair strand is disposed entirely within the sheath.

14. The suture construct of claim 11, wherein the shuttle has a fixed loop at a distal end.

15. A method of forming a suture construct from a structure braided with a pattern having a non-bifurcated segment and a bifurcated segment, the method comprising:
trimming a first length of the bifurcated segment to form a first distal end portion having a first opening;
trimming a second length of the bifurcated segment to form a second distal end portion having a second opening;
inserting a repair strand into the bifurcated segment by extending a length of the repair strand through the first distal end portion; and
inserting an activation strand into the bifurcated segment by extending the activation strand through the first opening and the second opening to tether the first distal end portion and the second distal end portion.

16. The method of claim 15, wherein the length of the repair strand extends through the first opening.

17. The method of claim 15, wherein the pattern has a second bifurcated segment, the method further comprising:
trimming a first length of the second bifurcated segment and a second length of the second bifurcated segment; and
splicing the second length of the second bifurcated segment into the first length of the second bifurcated segment.

18. The method of claim 17, further comprising trimming the first length of the second bifurcated segment or the second length of the second bifurcated segment after the splicing.

19. The method of claim 15, further comprising inserting a second length of the repair strand into the bifurcated segment by extending the second length of the repair strand through the second distal end portion.

20. The method of claim 15, further comprising splicing a shuttle through a portion of the length of the repair strand, wherein the portion of the repair strand is disposed entirely within the bifurcated segment when the repair strand is in the bifurcated segment.
